(19) United States
(12) Patent Application Publication
(10) Pub. No.: US 2019/0343951 A1
(43) Pub. Date: Nov. 14, 2019

(54) METHOD OF OPTIMIZING PEPTIDE IMMUNO-EPITOPE BY GLYCOSYLATION, OPTIMIZED PEPTIDE THEREOF AND ITS USE FOR CONJUGATE VACCINES

(71) Applicant: NATIONAL RESEARCH COUNCIL OF CANADA, OTTAWA (CA)

(72) Inventors: Wei ZOU, Ottawa (CA); Anne MARCIL, Pierrefonds (CA); Wangxue CHEN, Ottawa (CA); Robert PON, Gatineau (CA)

(21) Appl. No.: 16/477,218

(22) PCT Filed: Jan. 12, 2018
(86) PCT No.: PCT/CA2018/050030

§ 371 Date: Jul. 11, 2019

Related U.S. Application Data
(60) Provisional application No. 62/445,778, filed on Jan. 13, 2017.

(51) Int. Cl. A61K 39/385; A61K 39/145; A61K 39/00; A61K 47/54; A61P 35/00; A61P 31/16

(52) U.S. Cl. CPC A61K 39/385; A61K 39/145; A61K 39/001106; A61K 47/549; C12N 2760/16134; A61P 31/16; A61K 2039/6037; A61K 2039/6081; A61P 35/00

(57) ABSTRACT

The present invention provides a vaccine comprising a peptide antigen, one terminal end being coupled to a carrier molecule, and the other terminal end of the peptide being linked to a non-immunogenic moiety such as a saccharide. The thus terminally-glycosylated conjugated-peptide provides better immune responses in mice, able to generate unique mouse antibodies specific to the core region of these linear peptide epitopes. Results from immunization of both BALB/c and A/J strain mice revealed that the terminal glycosylation led to better antibody response towards the central epitope.

### FIELD OF THE INVENTION

The present invention relates to a glycosylated conjugated peptide vaccine having enhanced targeted immunogenicity and efficacies i.e. provoking an enhanced immune response.

### BACKGROUND OF THE INVENTION

Protein/large peptide-based cancer vaccine development has been hurdled by poor and unpredictable immune responses in mammals. The antibodies elicited by whole cancer cell antigens and large peptide/proteins often lead to antibodies having diverse effects, some inhibiting the cancer cell growth, some ineffective, while others stimulating it. Thus, so far, the cancer therapeutics are focused on (or limited to) monoclonal antibodies by separating “inhibitory” from “stimulatory” antibodies.¹ This approach, although effective, is very expensive and carries a big economic burden to our healthcare system.

Active immunization (vaccination) represents an alternative to the use of passive monoclonal antibody (mAb) therapy. However, the problem is that, in addition to poor immunogenicity and induced inappropriate immune responses, most epitopes are conformational, thus making vaccine construction extremely inconsistent and unpredictable.

With respect to vaccines against infectious disease such as seasonal and pandemic flu, it is highly desirable to have prophylactic vaccines with broad protection. Consequently, when so-called universal epitopes were sought on flu virus, the amino acid sequences of hemagglutinin² and neuraminidase³ were explored as vaccine candidates. However, the problem with that concept is that these conserved peptide epitopes are poorly immunogenic and fail to raise specific antibodies in mouse,^{2,3,4} thus limiting the potential use of these peptide epitopes as universal vaccine candidates.

To better manipulate the antibody response for therapeutic benefit, it is thus advantageous to utilise linear peptide epitopes that can predictably induce inhibitory antibodies. However, current data available suggest that raised antibodies are more likely to recognize terminal structures of the peptide.¹ These types of antibodies recognize a neoepitope (or artificial epitope) that is irrelevant for therapeutic or protective purposes.

In short, the problems are (1) poor immunogenicity of peptide epitope and (2) production of non-functional antibodies from undesired immuno-epitope.

Fusion of a peptide epitope to a protein or a large protein domain does not guarantee the production of relevant therapeutic antibodies^{3,5}. In the area of cancer vaccines, current cancer vaccines approved by FDA are only targeting cancer-causing bacteria/virus rather than cancer cells⁴. Other type of cancers may be treated by immunotherapy. However, the development of such biologics involves multiple engineering and characterization steps which take several years at extremely high costs from discovery to FDA approval.

With respect to a universal flu vaccine, universal anti-HA mAbs have been discovered through random screening of human serum,⁷ which recognize conformational epitopes. Recombinant trimeric HA stem region that was stabilised to adopt proper conformation has also been attempted as universal vaccine.⁸

At this time, specific antibodies against multiple strains of flu virus and certain cancers were generated by either extensive screening or by injecting animals with whole cell antigens. Yet, very few vaccines that provide broad protection against flu virus or specifically-inhibiting cancer cell growth have been approved by FDA. Effectivity, broad protection, and consistency are the parameters that are yet to be met.

### SUMMARY OF THE INVENTION

The present invention provides a novel peptide-conjugate with dramatically enhanced and targeted immunogenicity by “masking” the irrelevant end epitopes, thereby forcing the immune system to recognize the central and functional (i.e. effective) therapeutic epitope.

Therefore, in a first aspect, the present invention provides a vaccine comprising a peptide antigen comprising a first terminus and a second terminus, the first terminus being coupled to a carrier molecule, and the second terminus being linked to a non-immunogenic moiety (or vice versa).

In a second aspect, there is provided a composition comprising a vaccine as defined herein, in admixture with a saline solution, an adjuvant, an excipient, or a combination thereof.

A further aspect of the invention provides a method for optimizing immunogenicity of a peptide antigen in a peptide-conjugate vaccine, said peptide having (at least) two terminal-ends and a non-terminal region, the method comprising: selecting the peptide antigen; coupling the peptide antigen to a non-immunogenic moiety at one of the terminal-ends; and conjugating to a carrier protein at one of the terminal-ends of the peptide antigen, such that the non-immunogenic moiety blocks the one of the terminal-ends of the peptide antigen, thereby favouring an immune response against the non-terminal region thereof.

In a further aspect, there is provided a method for optimizing immunogenicity of a peptide antigen in a peptide-conjugate vaccine, said peptide comprising (at least) two terminal-ends and a non-terminal region, the method comprising: selecting a antigen-carrier conjugate comprising the peptide antigen conjugated to a carrier protein at one of the terminal-ends; and coupling a non-immunogenic moiety to an other terminal end of the peptide of the antigen-carrier conjugate, such that the non-immunogenic moiety blocks said other of the terminal-ends of the peptide antigen, thereby favouring an immune response against the non-terminal region thereof.

In a further aspect, the present invention provides a method for mounting an immune response against a non-terminal region of a peptide comprising (i.e. having at least) two terminal-ends, the method comprising: administering to a subject a vaccine as defined herein, wherein the subject is a mammal or a bird.

A further aspect of the invention provides a method for producing antibodies against a non-terminal region of a peptide comprising (i.e. having at least) two terminal-ends, the method comprising: administering to a non-human subject a vaccine as defined herein, wherein the non-human subject is a mammal or a bird.

There is also provided, in a further aspect, a method for preventing or treating an infection or a disease comprising the step of: administering to a subject a vaccine as defined herein, wherein the subject is a mammal or a bird.

In a further aspect, the present invention provides a method for immunizing a mammal or a bird against a disease and/or an influenza infection, comprising the step of: administering to a subject a vaccine as defined herein, wherein the subject is a mammal or a bird.

In a further aspect, the present invention provides use of a vaccine as defined herein, for the prevention or treatment of an infection or a disease in an animal.

In a further aspect, the present invention provides use of a vaccine as defined herein, for the immunization of an animal against a disease.

In a further aspect, the present invention provides use of a vaccine as defined herein, for the immunization of an animal against an influenza infection.

In a further aspect, the present invention provides a kit for immunizing a subject against a disease and/or an influenza infection, the kit comprising: a composition as defined herein; and a container.

### DESCRIPTION OF THE INVENTION

### Description of the Figures

FIG. 1A is a schematic diagram of one of the conjugate structures used for mouse immunizations. The peptide epitope is shown in a box; Lac-Ser is O-beta-lactosyl-serine; -S- is thio-Cys; KLH is Keyhole Limpet Hemocyanin.

FIG. 1B is a scheme representing the addition of non-immunogenic hydrophilic saccharide at one end of a linear peptide-conjugate.

FIG. 2 is a graph showing a titration curve of antisera directed towards Her2 protein from two distinct Lac-Her2 peptide antigens of SEQ ID No. 13 and No. 14.

FIGS. 3A-3C are FACS histograms of SKBR3 Her2-expressing cells labeled with (FIG. 3A) negative control; (FIG. 3B) Binding to SEQ ID No. 13 antisera; and (FIG. 3C) Binding to SEQ ID No. 14 antisera.

### ABBREVIATIONS

DT: diphteria toxin; HA: hemagluttinin; KLH: Keyhole limpet hemocyanin; NA: neuraminidase; TT: tetanus toxoid; Her2: Human epidermal growth factor receptor 2.

### Definitions

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%. When used in the context of a pH, the term “about” means +/−0.5 pH unit.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the terms “disease” may be used interchangeably or may be different in that the particular disorder, infection or condition may not have a known causative agent (so that etiology has not yet been worked out) and it is therefore not yet recognized as a disease but only as an undesirable condition or syndrome, wherein a more or less specific set of symptoms have been identified by clinicians.

The term “subject” as used herein refers to an animal, preferably a mammal or a bird, who is the object of administration, treatment, observation or experiment. “Mammal” includes humans and both domestic animals such as laboratory animals and household pets, (e.g. cats, dogs, swine, cattle, sheep, goats, horses, rabbits), and non-domestic animals such as wildlife, fowl, birds and the like. More particularly, the mammal is a rodent. Still, most particularly, the mammal is a human.

The molecule(s) described herein can be formulated as pharmaceutical compositions by formulation with additives such as pharmaceutically acceptable excipients, pharmaceutically acceptable carriers, and pharmaceutically acceptable vehicles.

As used herein, the term “pharmaceutically acceptable” refers to molecular entities and compositions that are physiologically tolerable and do not typically produce an allergic or similar unwanted reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans.

The term “adjuvant” refers to a diluent, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin.

The term “carrier molecule” as used herein means any protein that is foreign to the host receiving the vaccine and that triggers the immune system to mount a response. Particularly, in the case of the production of antibodies in rodents such as mice, conjugate vaccines use proteins as carrier molecules to increase the immunogenicity of small size antigens (e.g., peptides, oligosaccharides, polysaccharides and other haptens). These antigens are poor immunogens unless conjugated to proteins. The role of the carrier protein is to enhance immunogenicity by providing T-cell epitopes via MHC Class II presentation to T-helper cells. Carrier proteins both increase the magnitude of the immune response as well as engender B-cell “memory”, thus ensuring long-lasting immunity. In particular, large inactivated toxins such as tetanus toxoid, diphtheria toxoid and may be used as carriers in vaccines to develop strong immune responses. Other large proteins such as Keyhole Limpet Hemocyanin (KLH) are also well known to act as carrier molecule for immunogens. Alternatively, the carrier protein may be a human protein such as for example human serum albumin. The carrier molecule thus defined and claimed herein is therefore unlimited inasmuch as it provokes the desired immune response.

If administered as a medicinal preparation, the composition can be administered, either as a prophylaxis or treatment, to a patient by a number of methods. The present compositions may be administered alone or in combination with other pharmaceutical agents and can be combined with a physiologically acceptable carrier thereof. The effective amount and method of administration and aim of the present formulation can vary based on the individual subject, the stage of the disease or condition, and other factors apparent to one skilled in the art. In the case of a pharmaceutical formulation, during the course of the treatment, the concentration of the present compositions may be monitored (for example, blood antibody levels may be monitored) to ensure that the desired response is obtained.

### DETAILED DESCRIPTION OF PARTICULAR EMBODIMENTS

Because of their small size, peptides are often poorly immunogenic and do not induce a humoral immune response on their own. They can be made immunogenic by coupling them to a carrier molecule such as a protein. However, this may not work for all peptides.

Hereby proposed is the addition of non-immunogenic moiety at the end of a peptide-conjugate that may improve its interaction with antigen-presenting cells and induce a better immune response in animals, e.g. mice. Particularly, terminal glycosylation of a linear peptide epitope may point the immune response towards the core amino acid sequence rather than its terminal ones. Thus, vaccines were designed that selectively produce functional antibodies recognizing predefined epitope.

### Peptide Antigen

In a particular embodiment, the peptide antigen of the vaccine is any antigen peptide known in the art such as for example, peptide antigens derived from diseases caused by exogenous or endogenous factors, such as viral proteins, bacterial proteins or mammalian proteins. In the case, where the antigen peptide is derived from a human protein, such protein may, for example, be present in sick subjects but not present in normal healthy subjects (or present at lower levels), such as a cancer-specific antigen.

More particularly, the peptide antigen may comprise from 4 to 70 amino acids, particularly from 5 to 50 amino acids, more particularly from 6 to 30 amino acids, most particularly from 9 to 26 amino acids, still most preferably 4, 5, 6, 7, 8, 9, 10, 11, 12, 13 or 14 amino acids, as long as it is a relevant target (i.e. blocking or therapeutic) for the host immune response.

More particularly, the peptide antigen is chosen from a viral antigen that is a universal antigen, present on all strains of viruses. In particular, the viral antigen is a peptide derived from a protein from an influenza virus, such as for example, a peptide derived from influenza hemagglutinin (HA) or neuraminidase (NA).

More particularly, the HA or NA peptide antigen may comprise from 4 to 14 amino acids derived from the peptides: GLFGAIAGFIEGGW (SEQ ID No. 1) or ILRTQESEC (SEQ ID No. 2).

In particular, when the vaccine is an influenza vaccine, the peptide antigen may comprise the following sequence: GLFGAIAGFIEGGW (SEQ ID No. 1) or ILRTQESEC (SEQ ID No. 2).

In particular, when the vaccine is a cancer vaccine, the cancer-specific antigen is a breast cancer antigen. Particularly, the peptide antigen may originate from the Her2 antigen, such as, for example, a Her2 peptide. In particular, the Her2 antigen may be a peptide within the amino acid sequence (270-294): ALVTYNTDTFESMPNPEGRYTFGAS (SE ID No. 17). More specifically, the Her2 peptide antigen may comprise an amino acid sequence from 4 to 25 amino acids selected within the amino acid sequence (270-294): ALVTYNTDTFESMPNPEGRYTFGAS (SEQ ID No. 17).

Particularly, the Her2 peptide antigen may comprise an amino acid sequence from 4 to 13 amino acids selected within the amino acid sequences: ALVTYNTDTFES (SEQ ID No. 15) and MPNPEGRYTFGAS (SEQ ID No. 16). Specifically, the peptide antigen may comprise one of the following sequence: ALVTYNTDTFES (SEQ ID No. 15) and MPNPEGRYTFGAS (SEQ ID No. 16).

### Non-Immunogenic Moiety

In accordance with a particular embodiment of the present invention, the non-immunogenic moiety of the present vaccine of the invention is hydrophilic. More particularly, the non-immunogenic moiety may be selected from: a carbohydrate, a protein or a peptide. For example, the carbohydrate may be integrated into the peptide antigen by a terminal addition of: N-glycosyl asparagine, O-glycosyl serine or O-glycosyl threonine, or a combination thereof.

In accordance with a particular embodiment, the carbohydrate may be a monosaccharide or a disaccharide found in a human glycoprotein, more particularly, selected from: glucose, galactose, N-acetylglucosamine (GlcNAc), N-acetylgalactosamine (GalNAc), mannose, fucose, sialic acid, GlcNAc-GlcNAc, Glc-GalNAc, Gal-GalNAc, and lactose. According to a most particular embodiment, the disaccharide is lactose.

### Carrier Molecule

In accordance with a particular embodiment of the invention, the carrier molecule of the conjugate vaccine is a mammalian protein, such as for example human or bovine serum albumin.

According to an alternative embodiment, the carrier molecule is a large viral protein, preferably inactivated, such as, for example, diphtheria toxoid or Tetanus Toxoid (TT). Alternatively, the carrier molecule is Keyhole Limpet Hemocyanin (KLH).

### Conjugated Peptide Antigen

The present invention therefore provides a peptide antigen comprising a first terminus and a second terminus, the first terminus being coupled to a carrier molecule, and the second terminus being linked to a non-immunogenic moiety. Particularly, the non-immunogenic moiety is a carbohydrate or a saccharide or sugar.

Particularly, the sugar may be lactose.

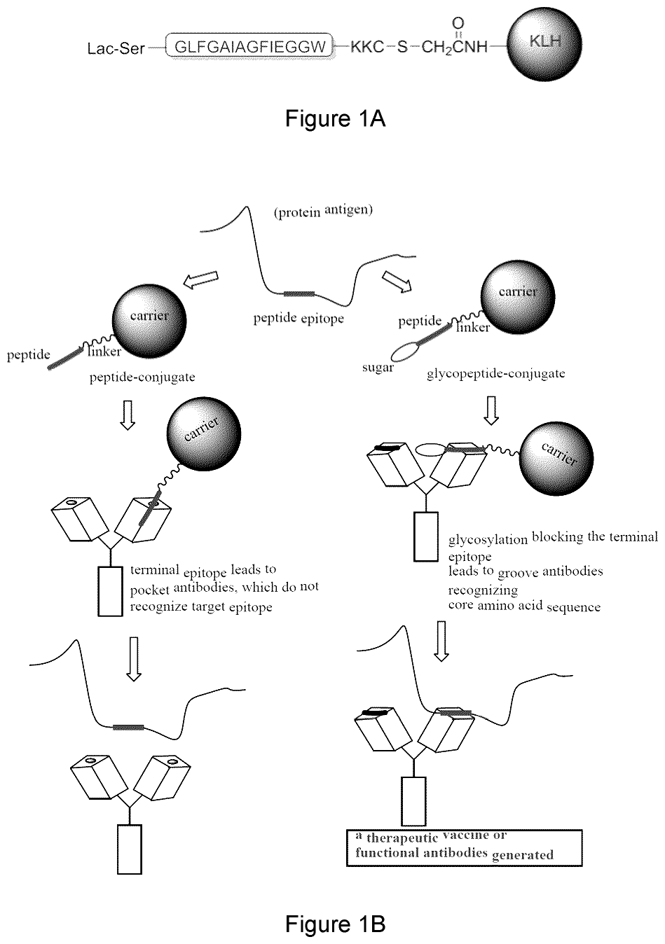

According to a particularly embodiment, the present invention provides a conjugated molecule generally represented by FIG. 1A, and particularly selected from the group consisting of:

| | SEQ |
|---|---|
| Antigen | ID |
| 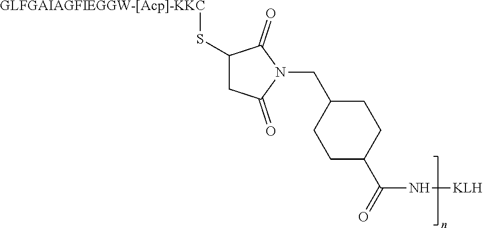 | No. 3 |
| 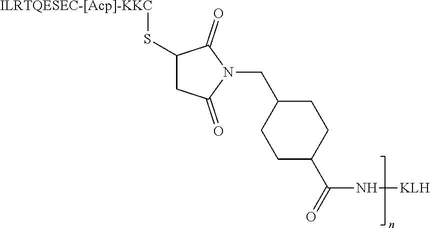 | No. 4 |
| 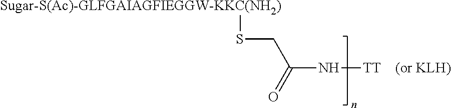 | No. 5 |
| 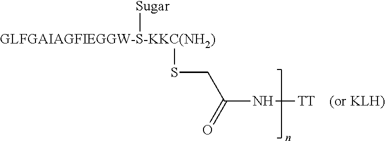 | No. 6 |
| 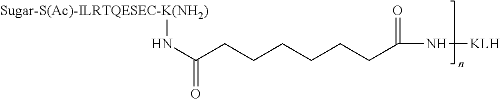 | No. 7 |
| 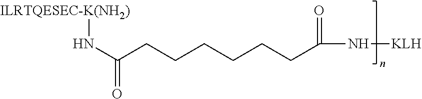 | No. 8 |
| 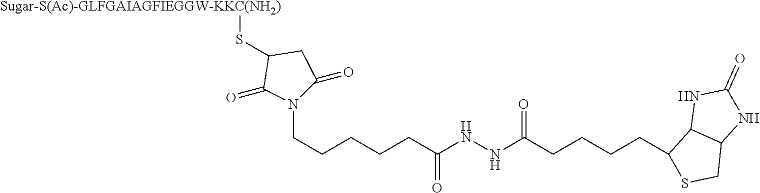 | No. 9 |
| 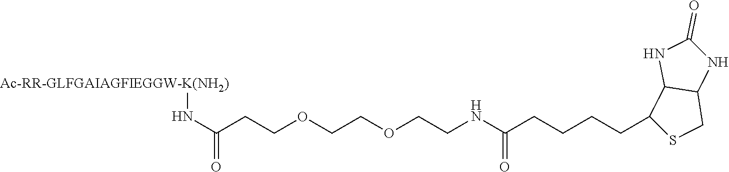 | No. 10 |
| 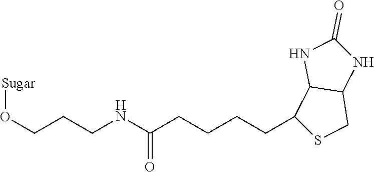 | Lactose |
| 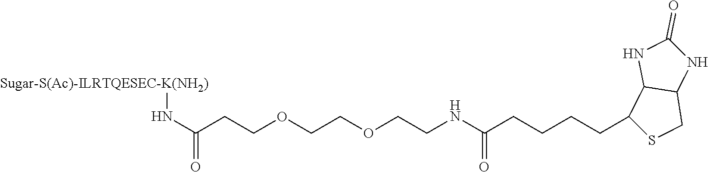 | No. 11 |
| 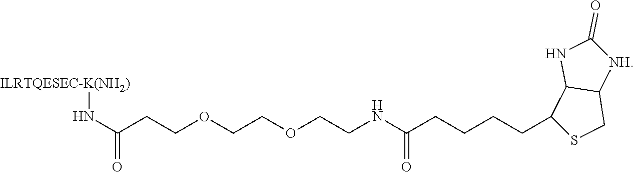 | No. 12 |
| 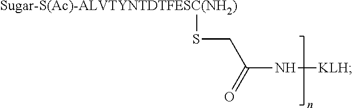 | No. 13 |
| 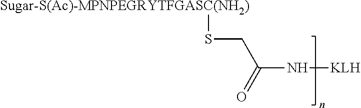 | No. 14 |

According to a further embodiment, the antigen consists essentially of SEQ ID No. 5 or No. 7:

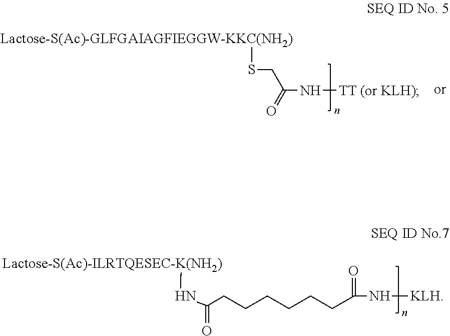

According to a further embodiment, the antigen consists essentially of SEQ ID No. 13 or No. 14):

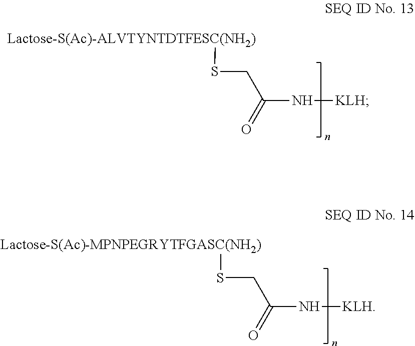

### Vaccine

Therefore, in accordance with a particular embodiment of the invention, there is provided the conjugate vaccine comprising a molecule chosen from: carbohydrate-HA-TT conjugate or a carbohydrate-HA-KLH conjugate, or carbohydrate-NA-KLH conjugate. Alternatively, the conjugate vaccine is: a carbohydrate-Her2peptide-KLH conjugate or a carbohydrate-Her2peptide-TT conjugate.

According to a further embodiment, the vaccine comprises a conjugated-peptide antigen selected from:

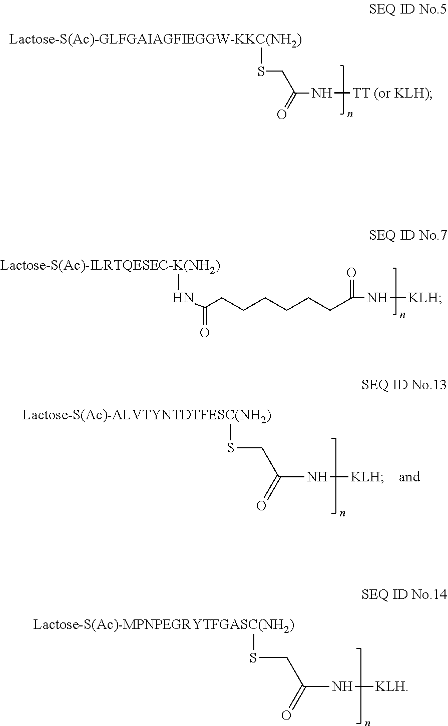

### Composition

In accordance with a particular embodiment, the invention also provides a composition comprising a vaccine as defined herein, in admixture with an adjuvant, an excipient, or a combination thereof. In particular, the excipient may be a saline solution.

### Methods

According to a particular embodiment, the invention provides a method for optimizing immunogenicity of a peptide antigen in a peptide-conjugate vaccine, said peptide having (at least) two terminal-ends and a non-terminal region, the method comprising: selecting the peptide antigen; coupling the peptide antigen to a non-immunogenic moiety at one of the terminal-ends; and conjugating to a carrier protein at an other of the terminal-ends of the peptide antigen, such that the non-immunogenic moiety blocks one (particularly, the other) of the terminal-ends of the peptide antigen, thereby favouring an immune response against the non-terminal region thereof.

Alternatively, there is provided a method for optimizing immunogenicity of a peptide antigen in a peptide-conjugate vaccine, said peptide having (at least) two terminal-ends and a non-terminal region, the method comprising: selecting the peptide antigen coupled to a non-immunogenic moiety at one of the terminal-ends; and conjugating to a carrier protein at the other terminal end of the peptide antigen, such that the non-immunogenic moiety blocks one (particularly the other) of the terminal-ends of the peptide antigen, thereby favouring an immune response against the non-terminal region thereof.

According to a particular embodiment, the invention provides a method for mounting an immune response against a non-terminal region of a peptide having (at least) two terminal-ends, the method comprising: administering to a subject a vaccine as defined herein, wherein the subject is a mammal or a bird. Particularly, the immune response is an antibody response, where particularly, antibodies binding the peptide antigen's non-terminal region are present, and antibodies against the peptide antigen's terminal-ends are substantially absent.

According to a particular embodiment, the invention provides a method for producing antibodies against a non-terminal region of a peptide having (at least) two terminal-ends, the method comprising: administering to a non-human subject a vaccine as defined herein, wherein the non-human subject is a mammal or a bird. Particularly, this method produces monoclonal antibodies or polyclonal antibodies.

In accordance with a particular embodiment, the invention provides a method for immunizing a mammal or a bird against an influenza infection, comprising the step of: administering to a subject a vaccine as defined herein, wherein the subject is a mammal or a bird. Particularly, the infection is an influenza viral infection or the disease is cancer.

### Method of Treatment

According to a particular embodiment, the invention provides a method for preventing or treating an infection or a disease comprising the step of: administering to a subject a vaccine as defined herein, wherein the subject is a mammal or a bird. Particularly, the mammal is a human and the infection is an influenza viral infection, or the disease is cancer.

### Uses

According to a particular embodiment, there is provided a use of a vaccine as defined herein, for the prevention or treatment of an infection or a disease in an animal.

According to a particular embodiment, the present invention provides a use of a vaccine as defined herein, for the immunization of an animal against an influenza infection or a disease, particularly where the animal is a mammal or a bird. More particularly, the infection is influenza or the disease is cancer.

### Kit

According to a particular embodiment, there is provided a kit for immunizing a subject against an infection or disease, the kit comprising: a composition as defined herein in a container. Particularly, the kit may further comprise instructions on how to administer said composition to the subject.

The following examples are put forth to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES—TERMINAL GLYCOSYLATION OF A LINEAR PEPTIDE EPITOPE FOR INCREASING IMMUNE RESPONSE

### Example 1—Material and Methods

The conjugates were prepared via a thio-ether bond between terminal Cys of the (glyco)peptide antigen and bromoacetyl KLH or TT (SEQ ID No. 5, 6, 13 & 14), or via an amide bond between terminal lysine amino group of (glycol)peptide antigen and the lysine amino groups on KLH or TT through a bivalent linker (e.g. disuccinimidyl substrate) (SEQ ID No. 7 & 8). The coating antigens are biotinylated (glycol)peptides prepared by coupling between terminal Cys or Lys (amino group) to maleimide biotin (No. 9) or biotin activated ester (lactose and SEQ ID No. 10, 12, 13).

Bromoacetylation of KLH: Typically, 20 mg of KLH (Sigma-Aldrich H7017) was solubilized in 2 mL of deionized water at room temperature, and buffer-exchanged to 10×PBS by an Amicon Ultra centrifugal filter (MWC 30K). To the above solution of KLH in 10×PBS (2 mL) was added 9 mg of bromoacetic acid N-hydroxysuccinimide ester in DMSO (0.18 mL) and incubated overnight at 4° C. The product was purified by a G-25 column (50×1.6 cm) with PBS as eluent and bromoacetyl KLH obtained was stored in PBS buffer. Similarly, bromoacetyl BSA was also made in parallel and MALDI indicated 9-10 bromoacetyl groups per BSA, we expect similar ratio of bromoacetyl group present in KLH.

### Conjugation Methods (FIG. 1B)

Method 1: (glyco)peptide antigen with terminal Cys and bromoacetyl KLH or TT are dissolved under the conditions of 0.1M phosphate buffer with 5 mM EDTA-0.01% sodium azide at pH 8.0-8.5 overnight at room temperature. As a reference to estimate the peptide antigen on KLH, bromoacetyl BSA (9-10 bromoacetyl per BSA) was also coupled with the peptide to give a conjugate with a ratio of peptide:BSA 6-7:1. The peptide antigen coupled to KLH carrier protein was presumed having a similar w/w ratio as BSA.

Method 2: (glyco)peptide antigen with terminal Lys was reacted with excessive amount of disuccinimidyl substrate in DMSO, resulting in a monosuccinimidyl substrate (glycol)peptide, which after purification was reacted with KLH in 10×PBS overnight at room temperature to afford the desired conjugates.

TABLE 1
Structures of peptide conjugate vaccines

| | | SEQ |
|---|---|---|
| Name | Vaccine | ID |
| HA | GLFGAIAGFIEGGW | No. 1 |
| NA | ILRTQESEC | No. 2 |
| HA- KLH | 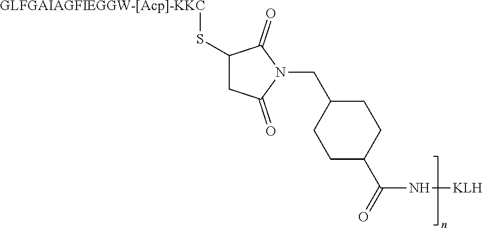 | No. 3 |
| NA- KLH | 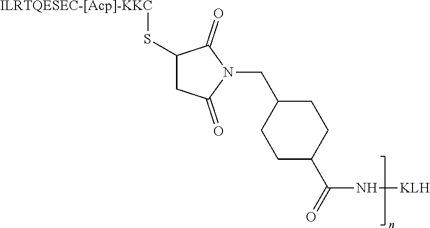 | No. 4 |
| Lac- HA-TT or Lac- HA- KLH | 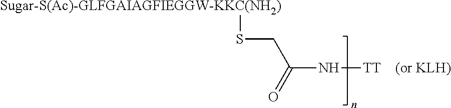 | No. 5 |
| HA- Lac-TT or HA- Lac- KLH | 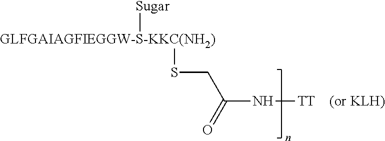 | No. 6 |
| Lac- NA- KLH | 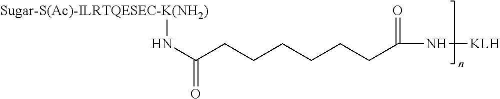 | No. 7 |
| NA- KLH | 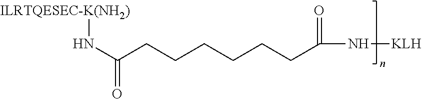 | No. 8 |
| Lac- Her2- KLH | 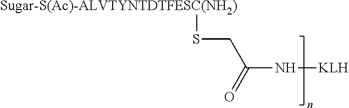 | No. 13 |
| Lac- Her2- KLH | 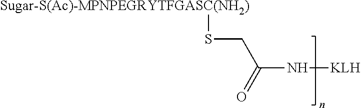 | No. 14 |

### Biotinylated Peptides

Typically, a (glyco)peptide antigen with terminal Cys was mixed with equivalent amount of biotin-maleimide (B1267, Sigma), or a (glyco)peptide antigen with terminal amine (Lys) with equivalent amount of biotin N-hydroxysuccinimide ester (H1759, Sigma) in DMSO at room temperature and the solution was kept for 5 hours, which was diluted with water and lyophilized to give product. The product was characterized by MALDI and no further purification was needed for ELISA.

TABLE 2
Structures of screening agents used in ELISA

| | | SEQ |
|---|---|---|
| Name | Structure | ID |
| Lac- HA | 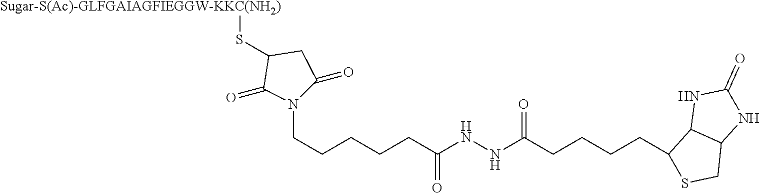 | No. 9 |
| X-HA | 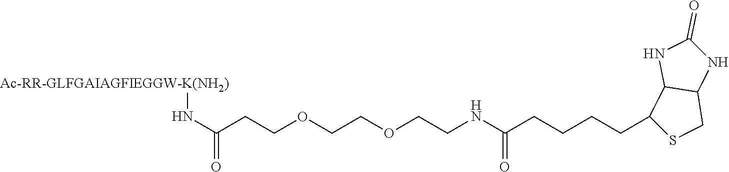 | No. 10 |
| Lac | 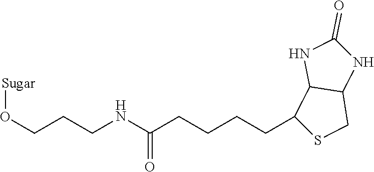 | |
| Lac- NA | 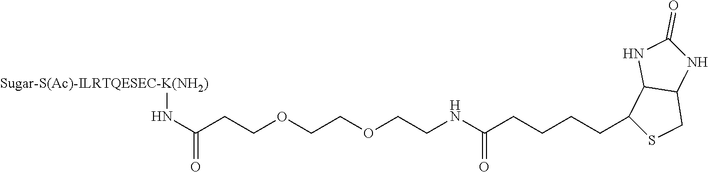 | No. 11 |
| NA | 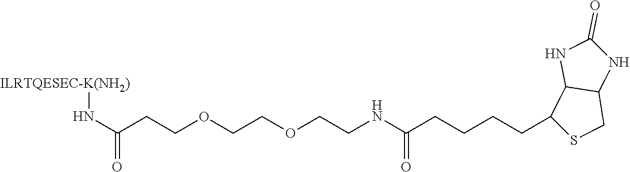 | No. 12 |

### Animal Immunization

Influenza HA and NA: Groups of 5-10 six to eight-week-old female BALB/c mice (Charles Rivers Laboratories, St. Constant, Quebec) were intraperitoneally injected with 100 μl of various HA vaccine preparations or controls (see Table 3) admixed with equal volume of alum at day 0, 14 and 21. At day 42, the mice were sacrificed for blood collection and serum preparation. All samples were stored at −80° C. until assay. Groups of 4 six-week old female A/J mice (The Jackson Laboratory, Bar Harbor, Me.) were injected intraperitoneally and subcutaneously with 100 μg of NA-KLH antigens emulsified in Titermax adjuvant (Cedarlane Labs, Burlington, ON) at day 0 and 21. Blood was collected in microvette CB 300Z (Sarstedt, Montreal, QC) at day 29, and serum was stored at −20° C. until further use.

Cancer Her2: Groups of 5-10 six to eight-week-old female BALB/c mice (Charles Rivers Laboratories, St. Constant, Quebec) were subcutaneously injected with various Her2 peptide vaccine preparations or controls admixed with c-di-GMP 10 μg/dose in PBS (0.1 mL) at day 0, 14 and 21. At day 35, the mice were sacrificed for blood collection and serum preparation. All samples were stored at −80° C. until assay.

### Example 2—HA-/NA- or Lac-Binding ELISA for Sera from HA-/NA-Peptide Conjugate Vaccines

Serum levels of HA- or Lac-specific IgG1 and IgG2a were measured by an enzyme-linked immunosorbent assay (ELISA). Briefly, 96-well Immunolon 2R microplates (Thermo Electron Corporation, Milford, Mass.) were coated with 0.2 μg rHA/well in 50 μl of bicarbonate buffer (pH 9.6) at 4° C. overnight. All the subsequent incubations were carried out at room temperature. The wells were blocked by incubation with 5% bovine serum albumin in PBS for 1 h, and then rinsed three times with PBS containing 0.05% Tween 20 (PBS-Tween). Duplicates of 100 μl pre-diluted samples (1:100) were added to the wells. After the plates were incubated for 3 h, alkaline phosphatase-conjugated goat antibodies specific for mouse IgG1 and IgG2a (Caltag Laboratories, Burlingame, Calif.) were added and incubated for 1 h. Color reactions were developed by the addition of p-nitrophenyl phosphate (pNPP) substrate (KPL, Inc., Gaithersburg, Md.), and optical density was measured at 405 nm with an automated ELISA plate reader (Synergy H1, Bio-Tek Instruments Inc, Winooski, Vt., USA). Pooled samples collected from mice that had been intranasally immunized with the rHA+CT or from the naïve mice were used as positive or negative controls for the assays, respectively. Pre- and post-immune sera titer of animals immunized with NA-KLH antigens were assessed by ELISA. Unless otherwise stated, all incubations were performed at room temperature. Briefly, half-area 96-well plates were coated with 25 μl per well of NeutrAvidin (Thermofisher, Rockford, Ill.) at 10 μg/ml in 50 mM carbonate buffer at pH 9.8. After a 2-hour incubation, microplates were washed three times in PBS and blocked for 30 min with 1% bovine serum albumin (BSA). Microplates were washed once with PBS and 25 μl of biotinylated peptide at 5 μg/ml was added and incubated overnight at 4° C. After 4 washes with PBS-Tween, 25 μl of serial dilutions of sera samples were added. After a 2-h incubation, microplates were washed 3 times with PBS-Tween and 25 μl of a 1/5,000 dilution of alkaline phosphatase conjugated goat anti-mouse IgG (H+L) (Jackson Immunoresearch, Cedarlane, Burlington, ON) in blocking buffer was added. After a 1-h incubation, microplates were washed 4 times and 25 μl of p-nitrophenyl phosphate (pNPP) substrate (Sigma-Aldrich Canada Co., Oakville, ON) at 1 mg/ml in carbonate buffer at pH 9.6 was added and further incubated for 30 min. Absorbance was read at 405 nm using a SpectraMax plate reader (Molecular Devices, Sunnyvale, Calif.).

TABLE 3
Sera titer of mice (Balb/c) immunized with HA peptides conjugated to TT

| | | Post-Immunization | | | | | |
|---|---|---|---|---|---|---|---|
| | | Titer determined on | | | | | |
| | Immunized | Lac-HA* | | X-HA* | | Lac | |
| Mouse | with | IgG1 | IgG2a | IgG1 | IgG2a | IgG1 | IgG2a |
| 1 | Lac-HA-TT* | >>1/6,400 | >1/6,400 | 1/1,600 | 1/100 | <1/100 | <1/100 |
| 2 | | >>1/6,400 | 1/6,400 | >>1/6,400 | <1/100 | <1/100 | <1/100 |
| 3 | | >>1/6,400 | >1/3,200 | >>1/6,400 | 1/400 | <1/100 | <1/100 |
| 4 | HA-Lac-TT* | 1/6,400 | >1/1,600 | <1/100 | <1/100 | <1/100 | <1/100 |
| 5 | | >1/6,400 | 1/400 | <1/800 | <1/100 | <1/100 | <1/100 |
| 6 | | 1/6,400 | >1/100 | 1/400 | <1/100 | <1/100 | <1/100 |

*see Table 1 and Table 2 for structures of vaccines and screening agents.

TABLE 4
Sera titer of mice (A/J) immunized with NA peptide conjugated to KLH

| | | Pre-immune sera | | Post-2ºimmunization | |
|---|---|---|---|---|---|
| | Immunized | Titer determined on | | Titer determined on | |
| Mouse | with | Lac-NA* | NA* | Lac-NA* | NA* |
| 1 | Lac-NA- | <1/100 | <1/100 | >>1/25,600 | >1/25,600 |
| 2 | KLH* | <1/100 | <1/100 | >>1/25,600 | >1/25,600 |
| 3 | | <1/100 | <1/100 | >1/25,600 | 1/25,600 |
| 4 | | <1/100 | <1/100 | >1/25,600 | 1/12,800 |
| 5 | NA- | <1/100 | <1/100 | 1/12,800 | >1/25,600 |
| 6 | KLH* | <1/100 | <1/100 | 1/25,600 | >1/25,600 |
| 7 | | <1/100 | <1/100 | <1/100 | >1/25,600 |
| 8 | | <1/100 | <1/100 | <1/100 | 1/25,600 |

*see Table 1 and Table 2 for structures of vaccines and screening agents respectively.

From the results of Tables 3 and 4, one can see that the conjugation to lactose increases dramatically the serum titer against the naked peptide.

### Example 3—Her2 Binding ELISA for Sera from Her2-Peptide Conjugate Vaccines (SEQ ID 14 and SEQ ID 15)

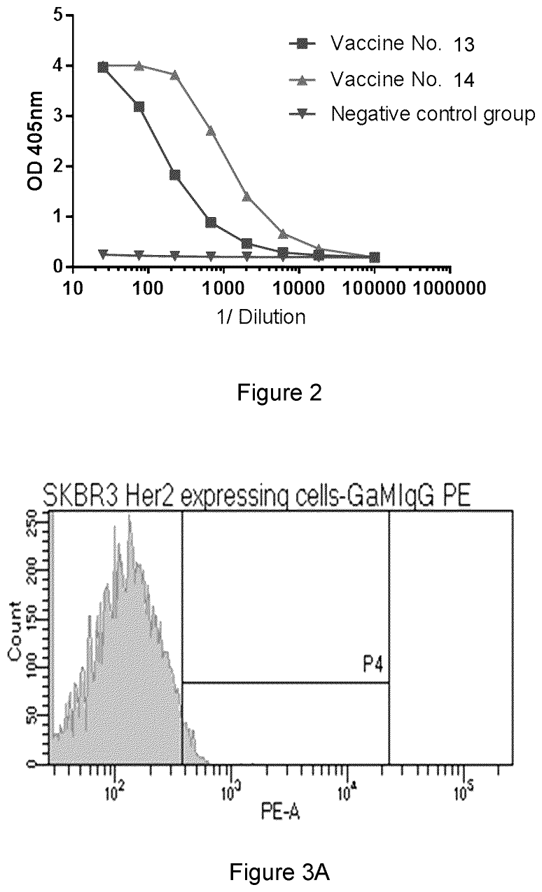

High binding ELISA plate wells (Immulon IV) were coated with 50 μl of recombinant human Her2 (0.00125 μg/μl PBS) for 18 h at 4° C. followed by removal and blocking with 100 μl of 1% skim milk in PBS for 1.5 h at room temperature. Wells were then washed 3×'s with PBS containing 0.05% Tween-20 followed by incubation at room temperature for 1 h with 3-fold dilutions of test mouse anti-sera (100 μl/well). Wells were subsequently washed 3×'s with PBS/Tween-20 and further treated with 100 μl of goat anti-mouse IgG (g)-alkaline phosphatase (1:2000 in 1% skim milk/PBS) for 1 h at room temperature. Following 3 washes with PBS/Tween-20 and a single PBS wash, freshly prepared p-nitrophenylphosphate substrate (100 ul) was added and the plates were allowed to develop for 10 minutes before quenching with 100 μl of 5% EDTA. Absorbance values were obtained at OD 405 nm using a multi-mode plate reader (FIG. 2). Endpoint titres were determined using negative control values+0.15 and 4 point curve fitting using GraphPad Prism.

### Example 4—FACS Analysis of Her2-Expressing Breast Carcinoma Cells Binding by Sera from Her2-Peptide Conjugate Vaccines (SEQ ID 13 and SEQ ID 14)

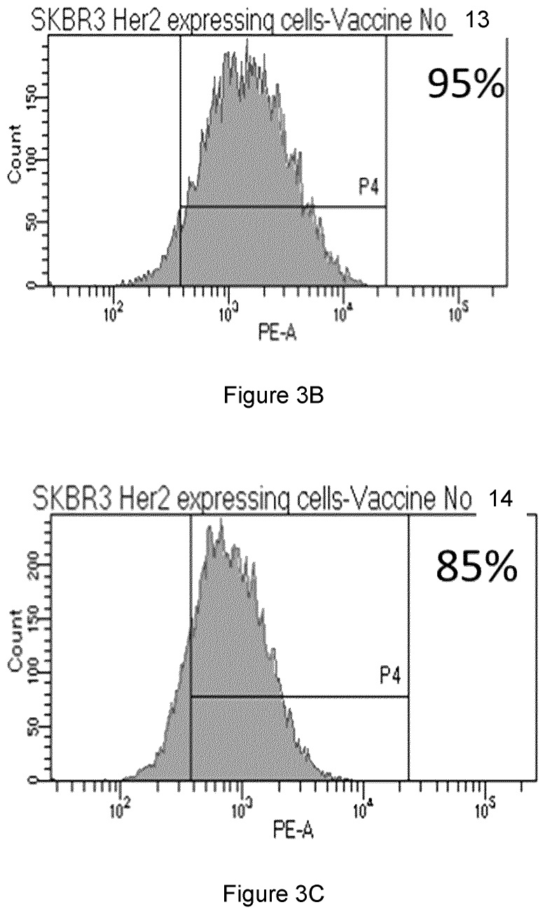

Human Her2 expressing breast carcinoma cells (SKBR3) were grown in RPMI-1640 media containing 10% FBS and harvested in log phase growth with trypsin/EDTA and washed with FACS staining buffer (1% BSA in PBS with 0.05% sodium azide. SKBR3 cells (0.5×10E6) were surface stained with 10 μl of the indicated mouse antisera for 30 minutes at 4° C. followed by washing with staining buffer and incubation for a further 30 minutes at 4° C. with goat anti-mouse IgG (H+L)-phycoerythrin. The negative control consisted of the goat anti-mouse IgG (H+L)-PE secondary antibody alone. Binding data was acquired on a BDFortessa flow cytometer collecting 10K live cell events, gating on the negative control, and the percent positive cells indicated in FIG. 3 histograms.

### Discussion

Because of their small size, peptides are often poorly immunogenic and do not induce a humoral immune response by their own. They can be made immunogenic by coupling them to a carrier protein. However, it may not be true for all peptide conjugates. Li et al have demonstrated that an influenza hemagglutinin-derived peptide conjugate (SEQ ID No. 3) was immunogenic in rabbit but not in Balb/c mouse.² Through careful examination of the peptide and its conjugate structures, we proposed that the poor immunogenicity may result from the high hydrophobicity of the peptide antigen. This can promote aggregation leading to poor immunogenicity and subsequent low humoral immunity. Thus, we proposed that the addition of non-immunogenic hydrophilic saccharide at the end of the peptide in a conjugate (SEQ ID No. 5) may improve its interaction with antigen-presenting B cells and induce a better immune response in animals, e.g. mice.

Furthermore, terminal glycosylation of a linear peptide epitope may also point the immune response towards the core amino acid sequence rather than terminal amino acids. Thus, we may be able to design a vaccine selectively producing functional antibodies recognizing predefined epitope.

To the best of our knowledge, the conjugate design as illustrated has never been reported. Apparently, this novel constituent and configuration of conjugates resulted in better immune responses in mice, and consequently novel (unique) mouse monoclonal antibodies specific to the linear peptide epitopes are generated. Results from immunization of both BALB/c and A/J strain mice also revealed that the terminal glycosylation indeed led to better antibody response towards epitope made of core amino acid sequence.

The significance of the invention is further validated through the vaccines based on glycosylated Her2 peptide antigens (SEQ ID No. 13 and 14). Antisera from these vaccines not only bind to Her2 proteins coated on ELISA plate as illustrated in FIG. 2, but also effectively bind to the Her2 expressing breast carcinoma cells as shown by the FASC analysis (see FIG. 3). This is a solid proof of concept, which may lead to wide applications in redesign of peptide based conjugate vaccines by terminal glycosylation for prophylactic or therapeutic purposes.

In short, the novelty of the proposed conjugate-antigens includes a) the improvement of linear peptide immunogenicity by terminal glycosylation, particularly, the hydrophobic peptides; and b) the conversion of immunodominance of a linear peptide from its terminal amino acids to other, more central regions of the peptide epitope (core amino acids).

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications and patent applications cited in this specification are herein incorporated by reference as if each individual publication or patent application were specifically and individually indicated to be incorporated by reference.

### REFERENCES

1. Yip, Y. L., Smith, G., Koch, J., et al, Identification of Epitope Regions Recognized by Tumor Inhibitory and Stimulatory Anti-ErbB-2 Monoclonal Antibodies: Implications for Vaccine Design. J. Immunol. 2001, 166, 5271-5278.
2. (a) Chun, S., Li, C., Domselaar, G. V., Wang, J., et al, Universal antibodies and their applications to the quantitative determination of virtually all subtypes of the influenza A viral hemagglutinins, Vaccine, 2008, 26, 6068-6076. (b) He, R et al. Influenza vaccine. US Patent (2014): U.S. Pat. No. 8,753,623.
3. Gravel, C., Li, C., Wang, J., et al, Qualitative and quantitative analyses of virtually all subtypes of influenza A and B viral neuraminidases using antibodies targeting the universally conserved sequences, Vaccine, 2010, 28, 5774-5784.
4. (a) Horvath A. et al. A hemagglutinin-based multipeptide construct elicit enhanced protective immune response in mice against influenza A virus infection. Immunol Lett. (1998 February); 60: 127-36. (b) Jackson D C and Brown L E. A synthetic peptide of influenza virus hemagglutinin as a model antigen and immunogen. Pept Res. (1991 May-June); 4:114-24. (c) Schoofs P G, et al. Epitopes of an influenza viral peptide recognized by antibody at single amino acid resolution. J Immunol. (1988 January); 140:611-6. (d) Nestorowicz A, et al. Antibodies elicited by influenza virus hemagglutinin fail to bind to synthetic peptides representing putative antigen sites. Mol Immunol. (1985 February); 22:145-54. (e) Bianchi E, et al. Universal influenza B vaccine based on the maturational cleavage site of the hemagglutanin precursor. J Virol. (2005 June); 79:7380-8.
5. Cancer Vaccines—National Cancer Institute. www.cancer.gov/about-cancer/causes-prevention/vaccines-fact-sheet.
6. Rockberg, J., Schwenk, J. M., Uhlen, M., Discovery of epitopes for targeting the human epidermal growth factor receptor 2 (HER2) with antibodies, Mol. Oncol., 2009, 3, 238-247.
7. (a) Corti, D., Voss, J., Gamblin, S. J., et al, A Neutralizing Antibody Selected from Plasma Cells That Binds to Group 1 and Group 2 Influenza A Hemagglutinins, Science, 2011, 333, 850-856. (b) Ekiert, D. C., Friesen, R. H., Bhabha, G., et al, A Highly Conserved Neutralizing Epitope on Group 2 Influenza A Viruses, Science, 2011, 333, 843-850. (c) Laursen, N. S., Wilson, I. A., Broadly neutralizing antibodies against influenza viruses, Antiviral Res. 2013, 98, 476-83.
8. Lu, Y., Welsh, J. P., Swartz, J. R. Production and stabilization of the trimeric influenza hemagglutinin stem domain for potentially broadly protective influenza vaccines, PNAS, 2014, 111, 125-130.

1. A vaccine comprising a peptide antigen comprising a first terminus and a second terminus, the first terminus being coupled to a non-immunogenic moiety, and the second terminus being linked to a carrier molecule.

2. (canceled)

3. The vaccine of claim 1, wherein the non-immunogenic moiety is a monosaccharide or disaccharide found in a human glycoprotein.

4. (canceled)

5. (canceled)

6. The vaccine of claim 3, wherein the monosaccharide or disaccharide is selected from the group consisting of: glucose (Glc), galactose (Gal), N-acetylglucosamine (GlcNAc), N-acetylgalactosamine (GalNAc), mannose, fucose, sialic acid, GlcNAc-GlcNAc, Glc-GalNAc, Gal-GalNAc, and lactose.

7. (canceled)

8. (canceled)

9. (canceled)

10. The vaccine of claim 1, wherein the peptide antigen is derived from an influenza virus protein.

11. (canceled)

12. The vaccine of claim 10, wherein the peptide antigen comprises a peptide derived from influenza hemagglutinin (HA) or from influenza neuraminidase (NA).

13. (canceled)

14. (canceled)

15. (canceled)

16. (canceled)

17. The vaccine of claim 12, wherein the vaccine comprises a carbohydrate-HA-tetanus toxoid (TT) conjugate molecule, a carbohydrate-HA-Keyhole Limpet Hemocyanin (KLH) conjugate molecule, or a carbohydrate-NA-KLH conjugate molecule.

18. The vaccine of claim 17, wherein the HA antigen comprises the amino acid sequence GLFGAIAGFIEGGW (SEQ ID NO. 1) or a sequence comprising from 4 to 13 contiguous amino acids thereof, or the NA antigen comprises the amino acid sequence ILRTQESEC (SEQ ID NO. 2) or a sequence comprising from 4 to 8 contiguous amino acids thereof.

19. The vaccine of claim 17, wherein the vaccine comprises:

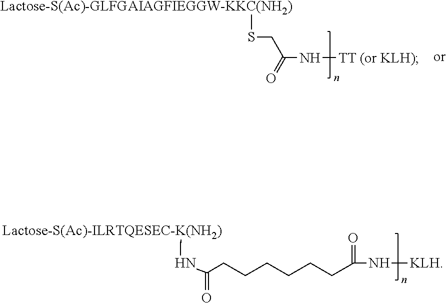

20. (canceled)

21. (canceled)

22. (canceled)

23. The vaccine of claim 1, wherein the peptide antigen is derived from the human cancer-associated protein Her2.

24. The vaccine of claim 23, wherein the Her2 antigen comprises a sequence of 4 to 24 contiguous amino acids from the amino acid sequence ALVTYNTDTFESMPNPEGRYTFGAS (SEQ ID No. 17), a sequence of 4 to 13 contiguous amino acids from the amino acid sequence ALVTYNTDTFES (SEQ ID No. 15), or a sequence of 4 to 13 contiguous amino acids from the amino acid sequence MPNPEGRYTFGAS (SEQ ID No. 16).

25. (canceled)

26. (canceled)

27. The vaccine of claim 24, wherein the vaccine comprises:

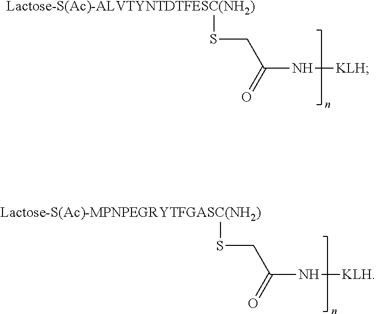

28. A composition comprising a vaccine as defined in claim 1, in admixture with a saline solution, an adjuvant, an excipient, or a combination of two or more thereof.

29. A method for optimizing immunogenicity of a peptide antigen in a peptide-conjugate vaccine, said peptide comprising a first terminus, a second terminus, and a non-terminal region, the method comprising:
selecting the peptide antigen;
coupling the peptide antigen to a non-immunogenic moiety at the first terminus; and
conjugating a carrier protein to the second terminus of the peptide antigen,
such that the non-immunogenic moiety blocks the first terminus of the peptide antigen, thereby favouring an immune response against the non-terminal region of the peptide antigen.

30. A method for optimizing immunogenicity of a peptide antigen in a peptide-conjugate vaccine, said peptide comprising a first terminus, a second terminus, and a non-terminal region, the method comprising:
selecting an antigen-carrier conjugate comprising the peptide antigen conjugated to a carrier protein at the second terminus of the peptide; and
coupling a non-immunogenic moiety to the first terminus of the peptide,
such that the non-immunogenic moiety blocks the first terminus of the peptide antigen, thereby favouring an immune response against the non-terminal region of the peptide antigen.

31. (canceled)

32. (canceled)

33. (canceled)

34. The method of claim 30, wherein the carbohydrate is a monosaccharide or disaccharide found in a human glycoprotein.

35. (canceled)

36. (canceled)

37. (canceled)

38. (canceled)

39. (canceled)

40. The method of claim 30, wherein the peptide antigen is derived from an influenza virus protein.

41. The method of claim 40, wherein the peptide antigen comprises a peptide derived from influenza hemagglutinin (HA) or from influenza neuraminidase (NA).

42. (canceled)

43. (canceled)

44. (canceled)

45. (canceled)

46. (canceled)

47. The method of claim 41, wherein the HA antigen comprises the amino acid sequence GLFGAIAGFIEGGW (SEQ ID No. 1) or the NA antigen comprises the amino acid sequence ILRTQESEC (SEQ ID No. 2).

48. The method of claim 47 wherein the antigen consists essentially of:

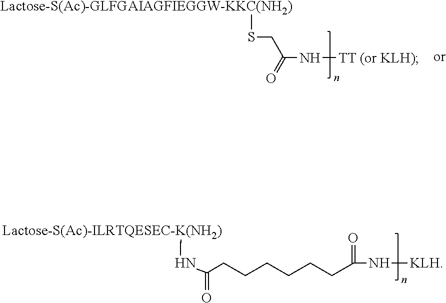

49. (canceled)

50. (canceled)

51. (canceled)

52. The method of claim 30, wherein the peptide antigen is derived from the human cancer-associated antigen Her2.

53. (canceled)

54. The method of claim 52, wherein the Her2 antigen comprises a peptide having a sequence of from 4 to 24 contiguous amino acids from the amino acid sequence ALVTYNTDTFESMPNPEGRYTFGAS (SEQ ID No. 17), having a sequence of from 4 to 13 contiguous amino acids from the amino acid sequence ALVTYNTDTFES (SEQ ID No. 15), or having a sequence of from 4 to 13 contiguous amino acids from the amino acid sequence MPNPEGRYTFGAS (SEQ ID No. 16).

55. (canceled)

56. The method of claim 52, wherein the Her2 antigen comprises:

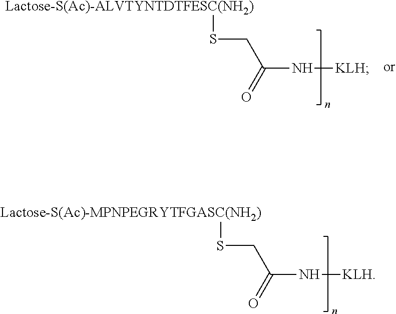

57. A method for mounting an immune response against a non-terminal region of a peptide having two terminal-ends, the method comprising: administering to a subject a vaccine according to claim 1, wherein the subject is a mammal or a bird.

58. (canceled)

59. (canceled)

60. (canceled)

61. (canceled)

62. (canceled)

63. A method for preventing or treating an infection or a disease comprising the step of: administering to a subject a vaccine according to claim 1, wherein the subject is a mammal or a bird.

64. The method of claim 63, wherein said infection or disease is influenza or cancer.

65. (canceled)

66. (canceled)

67. (canceled)

68. (canceled)

69. (canceled)

70. (canceled)

71. (canceled)

72. (canceled)

73. A kit for immunizing a subject against an influenza infection, the kit comprising:
a composition as defined in claim 28; and
a container.

74. (canceled)


SEQUENCE LISTING (17 sequences)

SEQ ID NO: 1 - protein, 14 aa, Artificial Sequence
  HA peptide antigen from the Influenza virus
GLFGAIAGFIEGGW

SEQ ID NO: 2 - protein, 9 aa, Artificial Sequence
  NA peptide antigen from the Influenza virus
ILRTQESEC

SEQ ID NO: 3 - protein, 17 aa, Artificial Sequence
  Conjugated peptide from the Influenza virus
  MISC_FEATURE (17)..(17): Wherein X represents a modified cysteine conjugated with KLH via a linker
GLFGAIAGFIEGGWKKX

SEQ ID NO: 4 - protein, 12 aa, Artificial Sequence
  Conjugated peptide from the Influenza virus
  MISC_FEATURE (12)..(12): Wherein X represents a modified cysteine conjugated with TT (or KLH) via a linker
ILRTQESECKKX

SEQ ID NO: 5 - protein, 18 aa, Artificial Sequence
  Conjugated peptide from the Influenza virus
  MISC_FEATURE (1)..(1): Wherein X represents a modified serine coupled with a sugar
  MISC_FEATURE (18)..(18): Wherein X represents a modified cysteine conjugated with TT or KLH via a linker
XGLFGAIAGFIEGGWKKX

SEQ ID NO: 6 - protein, 18 aa, Artificial Sequence
  Conjugated peptide from the Influenza virus
  MISC_FEATURE (15)..(15): Wherein X represents a modified serine coupled with a sugar
  MISC_FEATURE (18)..(18): Wherein X represents a modified cysteine conjugated with TT or KLH via a linker
GLFGAIAGFIEGGWXKKX

SEQ ID NO: 7 - protein, 11 aa, Artificial Sequence
  Conjugated peptide from the Influenza virus
  MISC_FEATURE (1)..(1): Wherein X represents a modified serine coupled with a sugar
  MISC_FEATURE (11)..(11): Wherein X represents a modified lysine conjugated with KLH via a linker
XILRTQESECX

SEQ ID NO: 8 - protein, 10 aa, Artificial Sequence
  Conjugated peptide from the Influenza virus
  MISC_FEATURE (10)..(10): Wherein X represents a modified lysine conjugated with KLH via a linker
ILRTQESECX

SEQ ID NO: 9 - protein, 18 aa, Artificial Sequence
  Conjugated peptide from the Influenza virus
  MISC_FEATURE (1)..(1): Wherein X represents a modified serine coupled with a sugar
  MISC_FEATURE (18)..(18): Wherein X represents a biotinylated cysteine
XGLFGAIAGFIEGGWKKX

SEQ ID NO: 10 - protein, 17 aa, Artificial Sequence
  Conjugated peptide from the Influenza virus
  MISC_FEATURE (17)..(17): Wherein X represents a biotinylated lysine
RRGLFGAIAGFIEGGWX

SEQ ID NO: 11 - protein, 11 aa, Artificial Sequence
  Conjugated peptide from the Influenza virus
  MISC_FEATURE (1)..(1): Wherein X represents a modified serine coupled with a sugar
  MISC_FEATURE (11)..(11): Wherein X represents a biotinylated lysine
XILRTQESECX

SEQ ID NO: 12 - protein, 10 aa, Artificial Sequence
  Conjugated peptide from the Influenza virus
  MISC_FEATURE (10)..(10): Wherein X represents a biotinylated lysine
ILRTQESECX

SEQ ID NO: 13 - protein, 14 aa, Artificial Sequence
  Conjugated peptide from the Human epidermal growth factor receptor 2 (HER2)
  MISC_FEATURE (1)..(1): Wherein X represents a modified serine coupled with a lactose
  MISC_FEATURE (14)..(14): Wherein X represents a modified cysteine conjugated with KLH via a linker
XALVTYNTDTFESX

SEQ ID NO: 14 - protein, 15 aa, Artificial Sequence
  Conjugated peptide from the Human epidermal growth factor receptor 2 (HER2)
  MISC_FEATURE (1)..(1): Wherein X represents a modified serine coupled with a lactose
  MISC_FEATURE (15)..(15): Wherein X represents a modified cysteine conjugated with KLH via a linker
XMPNPEGRYTFGASX

SEQ ID NO: 15 - protein, 12 aa, Artificial Sequence
  Peptide from the Human epidermal growth factor receptor 2 (HER2)
ALVTYNTDTFES

SEQ ID NO: 16 - protein, 13 aa, Artificial Sequence
  Peptide from the Human epidermal growth factor receptor 2 (HER2)
MPNPEGRYTFGAS

SEQ ID NO: 17 - protein, 25 aa, Artificial Sequence
  Peptide from the Human epidermal growth factor receptor 2 (HER2) - amino acids 270-294
ALVTYNTDTFESMPNPEGRYTFGAS